(19) United States
(12) Patent Application Publication
(10) Pub. No.: US 2011/0130678 A1
(43) Pub. Date: Jun. 2, 2011

(54) UNITARY SHAVE BIOPSY BLADE

(75) Inventor: Warren P. Williamson, IV, Loveland, OH (US)

(73) Assignee: CleveX, Inc., Columbus, OH (US)

(21) Appl. No.: 13/055,225

(22) PCT Filed: Jul. 29, 2009
(86) PCT No.: PCT/US09/52112

§ 371 Date: Jan. 21, 2011

Related U.S. Application Data
(60) Provisional application No. 61/085,017, filed on Jul. 31, 2008.

(51) Int. Cl. A61B 10/02

(52) U.S. Cl. 600/564

(57) ABSTRACT

A unitary shave biopsy blade comprises a first side edge, a second side edge laterally spaced from the first side edge, and a front edge extending laterally between the first and second side edges. A back edge extends laterally between the first and second side edges and is spaced longitudinally from the front edge. At least one of the front and back edges is at least partially sharpened to form a cutting edge. A top surface is defined laterally between the first and second side edges and longitudinally between the front and back edges. A bottom surface is defined laterally between the first and second side edges and longitudinally between the front and back edges and is spaced from the top surface by a blade thickness. At least one of the top and bottom surfaces includes a bend area feature which defines a local reduction in the blade thickness. A method of excising a target area from a surface of a skin tissue is also disclosed.

This application claims the benefit of U.S. Provisional Patent Application Ser. No. 61/085,017, filed Jul. 31, 2008 (pending), the disclosure of which is also fully incorporated herein by reference.

### TECHNICAL FIELD

The present invention relates to an apparatus and method for effecting shave biopsies.

### BACKGROUND OF THE INVENTION

In the practice of dermatology, lesions of questionable morphology may be removed and pathologically studied to determine if the lesion is cancerous or benign. There are many methods available to remove a skin lesion, whether for biopsy purposes or simply to excise the lesion from the patient. One common way to remove a skin lesion is by using a technique known as saucerization or “shaving.” This technique removes the lesion and superficial layers of the skin.

The shaving dermatological technique is a relatively simple procedure wherein a single edged blade is used to sever the lesion from the patient. While some doctors use scalpel blades, many instead use a single edged, razor-type blade. The razor type technique utilizes a flexible, flat surgical blade that is placed between a user's thumb and forefinger(s). Slight pressure is applied to the blade to induce the blade to curve away from the user's hand. By varying the finger pressure on the blade, the user can change the curvature of the blade to a certain extent. Commonly, a readily available double edge razor blade is used for this procedure and is snapped in half longitudinally to yield two sharp-edged half-blades for use. The patient's skin is pinched upward to produce a tight mound of skin, with the lesion located atop the mound. The curved edge may then be used in a sawing and/or sliding motion to scoop under the lesion, thus removing the lesion from the skin through use of a typically shallow and wide saucer-like cut.

Many millions of skin lesions are removed every year in the United States by this method. There are currently purpose-built shave blades which use various handles and holders to make the blade more user-friendly. Some, however, choose to use standard consumer razor blades, which are initially nonsterile and coated with a rust-resistant lubricant, and are typically made from 0.004″ thick chrome-plated carbon steel. The user bends the razor blade repeatedly along its long axis (which typically includes a series of mounting apertures, weakening that area of the blade), and eventually the razor blade breaks under the bending stress to create two long, thin, single-edged sharp excision blades. These two halves can then be sterilized and used on a patient.

These makeshift excision blades can present many challenges for the user. The correct blade curvature and cut depth are important to achieving a successful removal of the skin lesion without excessive removal of surrounding non-lesion skin. However, current blades only curve in a natural range of approximately 1″ radius at their tightest curvature, which may be inadequate for certain lesion removal situations. Another problem with the excision blades in current use is that the sharp razor blade edge extends across an entire width of the blade and the broken edges may leave sharp corners and surfaces elsewhere on the excision blade. Hence, the user must be cautious to avoid inadvertently cutting into her own fingertips during the lesion removal, in addition to the attention to the patient's skin required by the actual removal process. Moreover, the razor blades include no ergonomic features to facilitate safe use thereof. Finally, because the excision blades are not intended for bending during use, there may be a tendency for the blades to suddenly invert their curvature (in an “oil can” manner) in response to a slight change of force applied by the user. Such an unexpected shift in the blade configuration could be harmful to the patient and/or the user and could unnecessarily complicate the lesion removal process.

While various holders and guards have been proposed for use with double- or single-edged razor blades or excision blades formed as discussed above, these accessories add complexity and bulk to the lesion removal blade and can be relatively expensive and difficult to sterilize, particularly as compared to the simple, one-piece excision blades currently used.

### SUMMARY OF THE INVENTION

In an embodiment of the present invention, a unitary shave biopsy blade is described. The shave biopsy blade comprises a first side edge, a second side edge laterally spaced from the first side edge, and a front edge extending laterally between the first and second side edges. A back edge extends laterally between the first and second side edges and is spaced longitudinally from the front edge. At least one of the front and back edges is at least partially sharpened to form a cutting edge. A top surface is defined laterally between the first and second side edges and longitudinally between the front and back edges. A bottom surface is defined laterally between the first and second side edges and longitudinally between the front and back edges and is spaced from the top surface by a blade thickness. At least one of the top and bottom surfaces includes a bend area feature which defines a local reduction in the blade thickness. Another embodiment of the invention involves the construction and use of a kit of differently configured blades.

In an embodiment of the present invention, a method of excising a target area from a surface of a skin tissue is described. A unitary blade is provided, the unitary blade including longitudinally oriented and laterally spaced first and second side edges, laterally oriented and longitudinally spaced front and back edges, and oppositely disposed top and bottom surfaces, at least one of the front and back edges being at least partially sharpened to form a cutting edge spaced apart from both the first and second side edges, and at least one of the top and bottom surfaces including a bend area feature which defines a local reduction in a thickness of the unitary blade. A laterally directed compressive force is applied to the unitary blade to bend the unitary blade into a convex relationship with the surface of the skin tissue. The compressive force is adjusted to place the unitary blade into a predetermined bend profile. A shape of the bend profile is at least partially dependent upon a shape of the bend area feature. The bend profile includes at least a portion of the cutting edge. The target area is excised from the skin tissue through substantially longitudinally oriented motion of the unitary blade.

### BRIEF DESCRIPTION OF THE DRAWINGS

For a better understanding of the invention, reference may be made to the accompanying drawings, in which:

FIG. 1 is a perspective top view of one embodiment of the present invention having a first configuration;

FIG. 2 is a cross-sectional view taken along line 2-2 of FIG. 1;

FIG. 3 is a perspective top view of the embodiment of FIG. 1 having a second configuration;

FIG. 4 is a perspective top view of the embodiment of FIG. 1 having a third configuration;

FIG. 4A is a side elevational view of the embodiment of FIG. 4, but showing the bend profile as formed during use as a result of the bend area feature configuration shown in FIG. 4;

FIG. 5 is a perspective top view of the embodiment of FIG. 1 having a fourth configuration;

FIG. 5A is a side elevational view of the embodiment of FIG. 5, but showing the bend profile as formed during use as a result of the bend area feature configuration shown in FIG. 5;

FIG. 6 is a perspective top view of the embodiment of FIG. 1 having a fourth configuration;

FIG. 6A is a side elevational view of the embodiment of FIG. 6, but showing the bend profile as formed during use as a result of the bend area feature configuration shown in FIG. 6;

FIG. 7 is a perspective top view of the embodiment of FIG. 1 having a fifth configuration;

FIG. 7A is a side elevational view of the embodiment of FIG. 7, but showing the bend profile as formed during use as a result of the bend area feature configuration shown in FIG. 7;

FIG. 8 is a perspective top view of the embodiment of FIG. 1 having a sixth configuration;

FIG. 9 is a perspective bottom view of the embodiment of FIG. 1 having a seventh configuration;

FIGS. 10a and 10b depict an example sequence of operation of the embodiment of FIG. 1; and

FIG. 11 illustrates an example of a manufacturing environment of the embodiment of FIG. 1.

### DESCRIPTION OF EMBODIMENTS

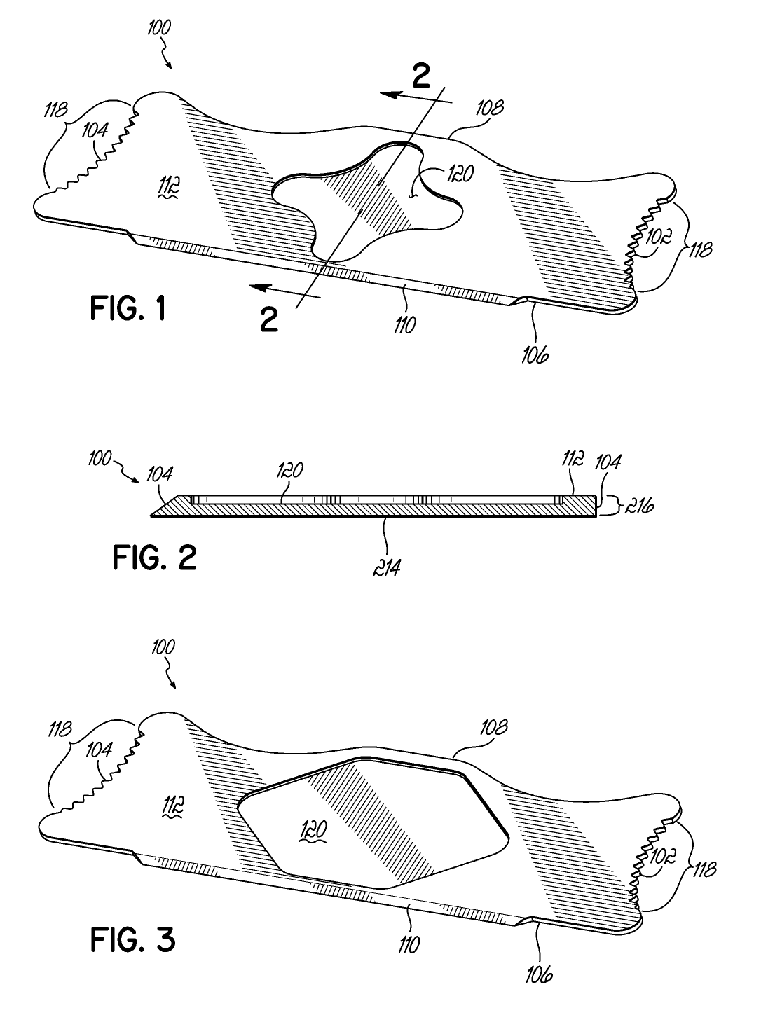

In accordance with the present invention, FIG. 1 depicts a unitary shave biopsy blade 100. “Unitary” here is used to mean that the shave biopsy blade 100 is formed and presented to the user as a single piece of material which has been shaped and treated to comprise the described structure but does not preclude the use of a laminated or other multi-piece blank from which the shave biopsy blade may be formed.

The shave biopsy blade 100 includes a first side edge 102, a second side edge 104 laterally spaced from the first side edge, a front edge 106 extending laterally between the first and second side edges, and a back edge 108 extending laterally between the first and second side edges and spaced longitudinally from the front edge. At least one of the front and back edges 106 and 108 is at least partially sharpened to form a cutting edge 110. Each cutting edge 110 may be laterally separated from the first and second side edges, as shown in FIG. 1, which may serve to space the cutting edge apart from the user's digits during use.

A top surface 112 is defined laterally between the first and second side edges 102 and 104 and longitudinally between the front and back edges 106 and 108. As seen in the cross-sectional view of FIG. 2, the shave biopsy blade 100 also includes a bottom surface 214, defined laterally between the first and second side edges 102 and 104 and longitudinally between the front and back edges 106 and 108. The bottom surface 214 is spaced from the top surface 112 by a blade thickness 216.

At least one of the first side edge 102, the second side edge 104, and the back edge 108 may be ergonomically contoured, as shown in FIG. 1, for contact with a digit of a user. An ergonomically contoured edge feature may assist the user in comfortably grasping the shave biopsy blade 100 and applying a bending force in a secure and repeatable manner, as will be described below. The edge feature, or any other structure of the shave biopsy blade 100, may be bent or otherwise offset from other portions or structures of the shave biopsy blade. However, for simplicity, the shave biopsy blade 100 will be shown and described herein as having a substantially planar configuration when in a resting state.

Whether or not an ergonomic contour is present, at least one of the first side edge 102, the second side edge 104, and the back edge 108 may include a friction-increasing feature 120 along at least a portion thereof, for engagement with a digit of a user. For example, and as shown, a plurality of serrations or scallops may serve as a friction-increasing feature 120 and help the user to maintain a firm grip on the shave biopsy blade 100.

As can be seen in both FIGS. 1 and 2, at least one of the top and bottom surfaces 112 and 214 includes a bend area feature 120 which defines a local reduction in the blade thickness 216. For example, the bend area feature 120 may be etched into the top and/or bottom surfaces 112 and 214. The bend area feature 120 may be formed in any manner, however, and may be any suitable size or shape. For example, the bend area feature 120 might extend into approximately half of the depth of the blade thickness 216 into the top surface 112. As another example, the bend area feature 120 could instead extend entirely through the blade thickness 216 as a slit, multiple slits, or other type(s) of aperture(s).

The bend area feature 120 may be spaced apart from the first and second side edges 102 and 104 and the front and back edges 106 and 108, as shown, or may intersect with one or more of the first and second side edges and the front and back edges. The bend area feature 120 may have a footprint shape operative to facilitate at least one desired bending characteristic in the shave biopsy blade 100 upon application of compressive force in the lateral direction to the shave biopsy blade.

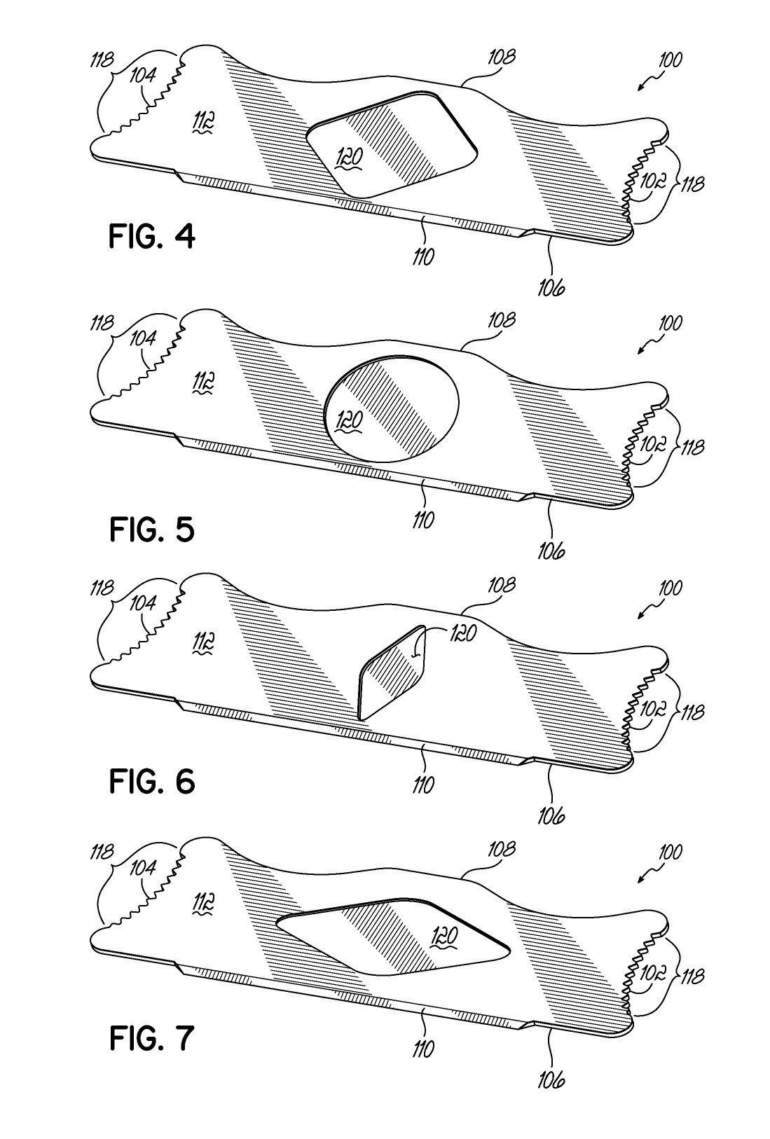

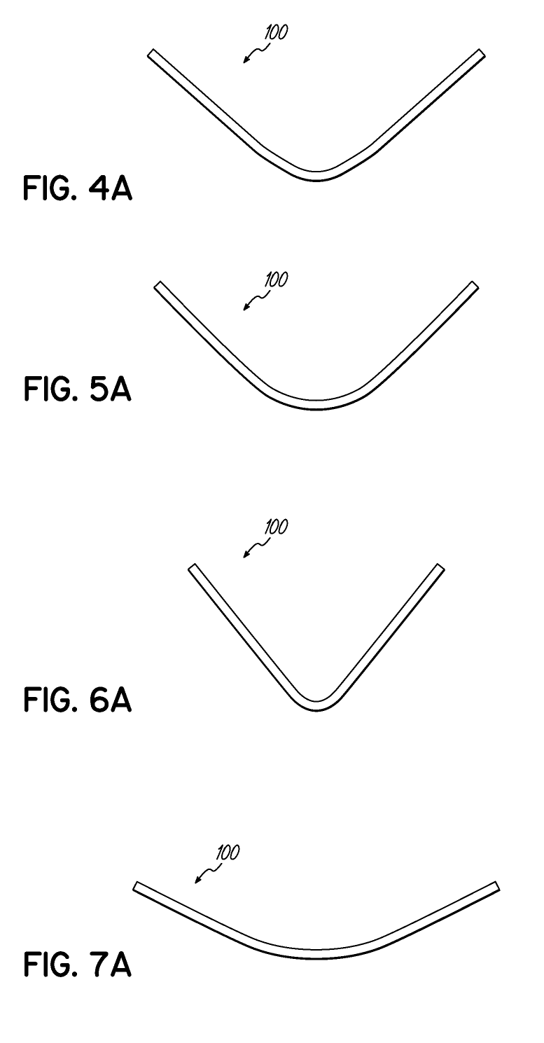

Examples of some possible configurations of footprint shapes of bend area features 120 are shown in FIGS. 1 and 3-9. The bend area feature 120 in FIG. 1 has a star-like shape. The bend area feature 120 in FIG. 3 has a hexagonal shape. The bend area feature 120 in FIG. 4 has a square shape. The bend area 120 in FIG. 5 has a circular shape. The bend area feature 120 in FIG. 6 has an upright diamond shape. The bend area feature 120 in FIG. 7 has a flattened diamond shape.

The respective bend profiles of the blades 100 shown in FIGS. 4, 5, 6 and 7 are illustrated in FIGS. 4A, 5A, 6A and 7A. These bend profiles are achieved during use by using the thumb and forefinger to bend the normally planar blade. The bend profiles achieved in FIGS. 4A, 5A, 6A and 7A are the result of applying essentially the same bending force to each blade. It will be appreciated that the configuration and design of the bend area feature 120 determines the bend profile assuming the application of a constant bending force.

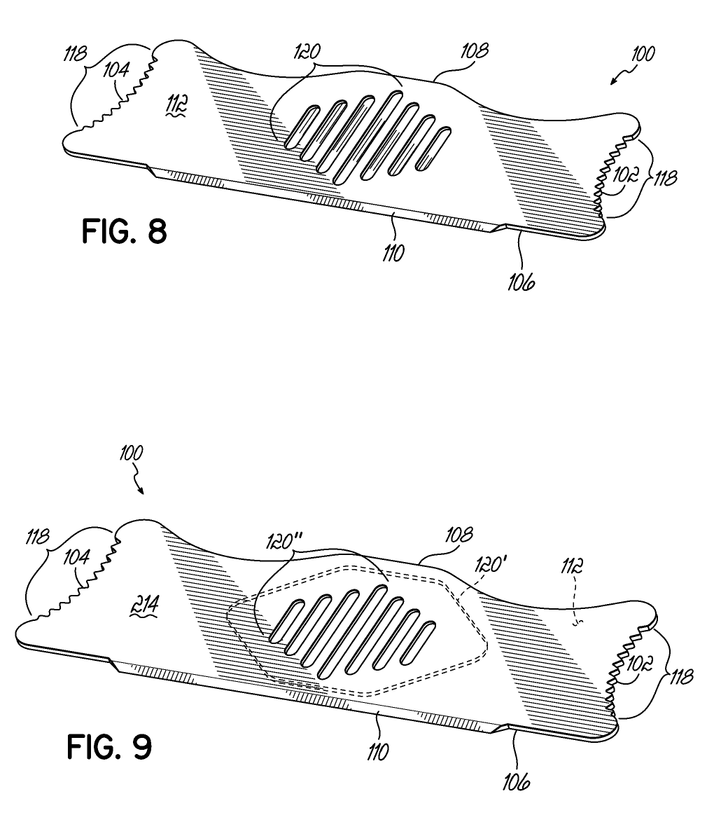

In FIG. 8, the bend area feature 120 is made up of a plurality of slits, which each may have any suitable size, shape, orientation, or other characteristics and need not match one another. The slits, when present, can assist in allowing the bend area feature 120 to extend completely through the blade thickness 216 while preventing entry and lodgement of the patient's skin tissue into the bend area feature 120, as might occur with a larger-area aperture as the shave biopsy blade drags across the skin during use.

FIG. 9, in contrast, is a bottom view of the shave biopsy blade 100 which includes two bend area features 120′ and 120″. The bend area feature 120′ has a hexagonal shape and is located on the top surface 112. The bend area feature 120″ includes a plurality of slots and is located on the bottom surface 214. Neither of the two bend area features 120′ and 120″ is shown as extending entirely through the blade thickness 216, though such a configuration is possible. The bend area feature 120″ located on the bottom surface 214 will contact the skin tissue during use of the shave biopsy blade 100 and provides reduced surface area and thus less friction between the bottom surface 214 and the skin tissue. The configuration of the bend area feature 120″ located on the bottom surface 214 in the embodiment of FIG. 9 is similar in concept and operation to the divoted blade of a santuko chef's knife.

Though the general shape of the shave biopsy blade 100 is the same in all of the depicted configurations, each of the different bend area features 120 shown can facilitate different bending characteristics of the shave biopsy blade as discussed above and shown, by way of example in FIGS. 4A-7A. One of ordinary skill in the art can readily provide and/or choose a shave biopsy blade 100 having a suitable bend area feature 120 (not limited to those shown in the Figures) for any particular application of the present invention. This may be achieved, for example, by providing a kit, which may or may not be contained in a single package, containing multiple blades 100 each having differently configured bend area features 120. For example, the respective bend area features may have different sizes, shapes, and/or amount of the local reduction in blade thickness. This allows the user to perform a method in accordance with one or more other aspects disclosed herein, by also choosing the appropriate blade and bend characteristics from the kit for the patient's needs. For example, a first blade having a first bend area feature or configuration 120 may be chosen and used to excise tissue at a first location on the patient's body, and a second blade having a second bend area feature or configuration 120 different from the first may be chosen and used to excise tissue at a second, different location on the patient's body. Alternatively, the first and second blades may be used to excise tissue at the same location on the patient's body, depending on the needs of the physician.

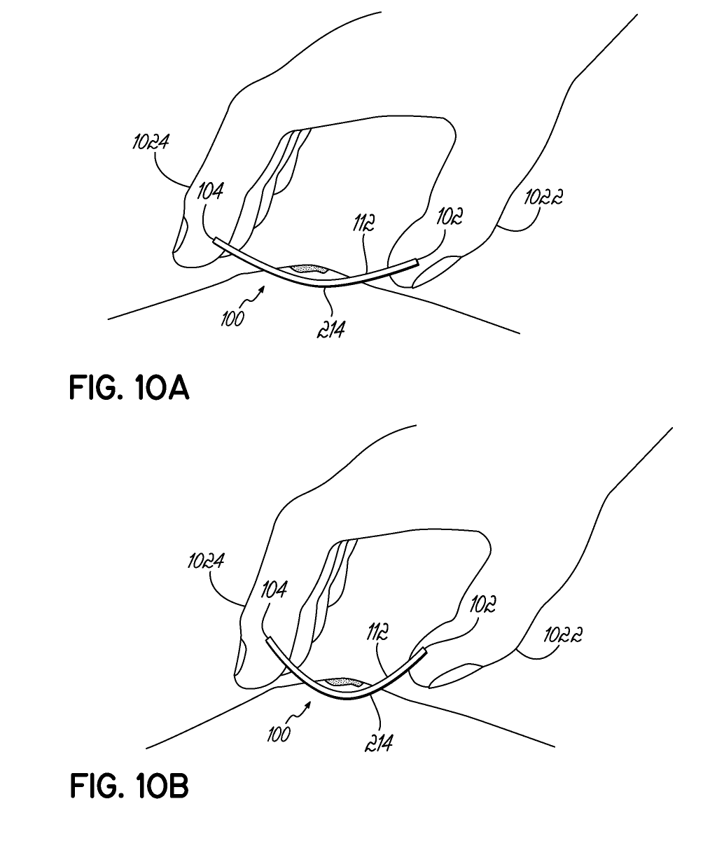

Regardless of the shape or configuration of the bend area feature 120, each shape biopsy blade 100 is configured to bend in a lateral direction under compressive force, as shown in FIGS. 10a-10b. In these Figures, a user is gripping the shape biopsy blade 100 with a thumb 1022 on the first side edge 102 and a forefinger 1024 on the second side edge 104. The front and back edges 106 and 108 are parallel with the plane of the drawing page in FIGS. 10a and 10b. The user can pinch the thumb 1022 and forefinger 1024 together to apply a laterally directed compressive force and thereby curve the shave biopsy blade 100 concavely away from the user's hand, as shown. Before or during the lesion removal procedure, the user can adjust the compressive force exerted upon the shave biopsy blade 100 and thereby selectively vary the profile of the bend of the shave biopsy blade. As shown in the sequence from FIG. 10a to FIG. 10b, for example, more compressive force could result in a steeper or more pronounced bend in the shave biopsy blade 100. The location and amount of bending of the shave biopsy blade 100 are responsive to at least one of the amount of compressive force applied; the relative configurations of the first and second side edges 102 and 104, the front and back edges 106 and 108, and the blade thickness 216; and a footprint shape of the bend area feature 120.

To use the shave biopsy blade 100 to excise a target area (e.g., a skin lesion and small amount of surrounding skin) from a surface of a skin tissue, the user may first select a desired shave biopsy blade 100 from a group of shave biopsy blades having differently configured bend area features 120, with the selection being based at least partially upon the predetermined bend profile of the selected shave biopsy blade (which is facilitated by that blade's bend area feature). For example, the user may wish to choose a shave biopsy blade 100 having a very sharply angled bend profile (FIG. 6A) to make a narrow, deep (“trough-like”) incision for one target area, while the bend profile desired for another target area might be one which produces a shallower, wider (“saucer-like”) incision (FIG. 7A). While a portion of the bend profile is dependent upon the compressive force exerted by the user, the bend area feature 120 also contributes materially to the way in which a particular shave biopsy blade 100 bends under the application of a constant bending force.

Next, the user grasps the selected shave biopsy blade 100, such as by placing the contacting the first and second side edges 102 and 104 in a substantially perpendicular fashion with the thumb 1022 and forefinger 1024, respectively, possibly with retention assistance by the friction-increasing features 118. The user then applies a laterally directed compressive force to the shave biopsy blade 100, to bend the shave biopsy blade into a convex relationship with the surface of the skin tissue. The compressive force applied by the user will place the shave biopsy blade 100 into a predetermined bend profile, as shown in the sequence of FIGS. 10a and 10b. The shape of the bend profile may be at least partially dependent upon the footprint shape and/or depth of the bend area feature 120. The bend profile should include at least a portion of the cutting edge 110.

Since the bend area feature 120 of the depicted shave biopsy blade 100 is substantially in line with the center of the sharpened cutting edge 110, the bend profile of the shave biopsy blade is easily controlled by the user through pressure on the first and second side edges 102 and 104. The sections of the shave biopsy blade 100 which are outside the bend area feature 120 are thicker, and thus stiffer and less yielding, than the bend area feature itself, which does most of the shape contouring. This arrangement assists a user in controlling the cutting depth and shave profile of the shave biopsy blade 100. It should be noted that the bend area feature 120 is not necessarily located in a centered position with respect to the cutting edge 110.

Once the desired bend profile is achieved, the user can then proceed to excise the target area from the skin tissue through substantially longitudinally oriented (into or out of the plane of FIGS. 10a and 10b) motion of the shave biopsy blade 100. In order to perform the desired excision, the user contacts the surface of the skin tissue adjacent the target area with the bend profile, and particularly the cutting edge 110 portion included in the bend profile. The user then applies a motive force to the shave biopsy blade 100. The generally longitudinally directed motive force may include some lateral aspects, giving the shave biopsy blade 100 a back and forth “sawing” motion as the blade proceeds longitudinally along and through the patient's skin, if desired by the user. For example, concurrently with holding the desired lateral compressive force, the user's thumb 1022 and forefinger 1024 could be used to push, pull, and/or saw back and forth to move the shave biopsy blade 100 toward the target area to be removed. The motive force could be transferred from the user's thumb 1022 and forefinger 1024 through the friction-increasing features on the first and second side edges 102 and 104. The motive force could also or instead be transferred from a digit of the user (not necessarily the thumb 1022 or forefinger 1024) to the shave biopsy blade 100 through pressure exerted upon the back edge 108 of the shave biopsy blade.

Through the motive force applied to at least one of the first and second side edges 102 and the back edge 108, the shave biopsy blade 100 can then be moved substantially longitudinally toward the target area. When the shave biopsy blade 100 has been brought to the desired cutting position, the user may then pass at least a portion of the bend profile (e.g., that portion having the cutting edge 110) through the skin tissue beneath and adjacent to the target area, in order to sever the target area with the cutting edge.

Optionally, the user may somewhat adjust the predetermined bend profile during excision of the target area by selectively varying the laterally directed compressive force exerted on the shave biopsy blade 100. Such adjustment may be desirable, for example, when the borders of the target area to be removed are uneven or the bend profile approaches an anatomical feature of the patient's skin which should remain undamaged.

Once the target area has been severed, the user may move the shave biopsy blade 100 substantially longitudinally away from the target area, though an upward or lifting motion of the shave biopsy blade may also be provided in certain use environments. Regardless of the motions employed, the shave biopsy blade 100 may be removed from the skin tissue once the target area has been removed.

It is contemplated that at least a portion of the shave biopsy blade 100 may include a drug-eluting material and/or feature. “Elute” is used herein to indicate that a therapeutic agent is released, leached, diffused, or otherwise provided to the target tissue. Therefore, at least a portion of the shave biopsy blade 100 could be adapted to elute a therapeutic agent, such as, but not limited to, an anesthetic, anti-inflammatory, coagulant, cauterizing agent, scar-reducing material, or antiseptic fluid, to the wound (not shown) created by removal of the target area from the skin tissue. Additionally, it is contemplated that at least a portion of the shave biopsy blade 100 may be coated with, elute, or otherwise include a lubricant which helps decrease friction of the shave biopsy blade against the patient's skin surface.

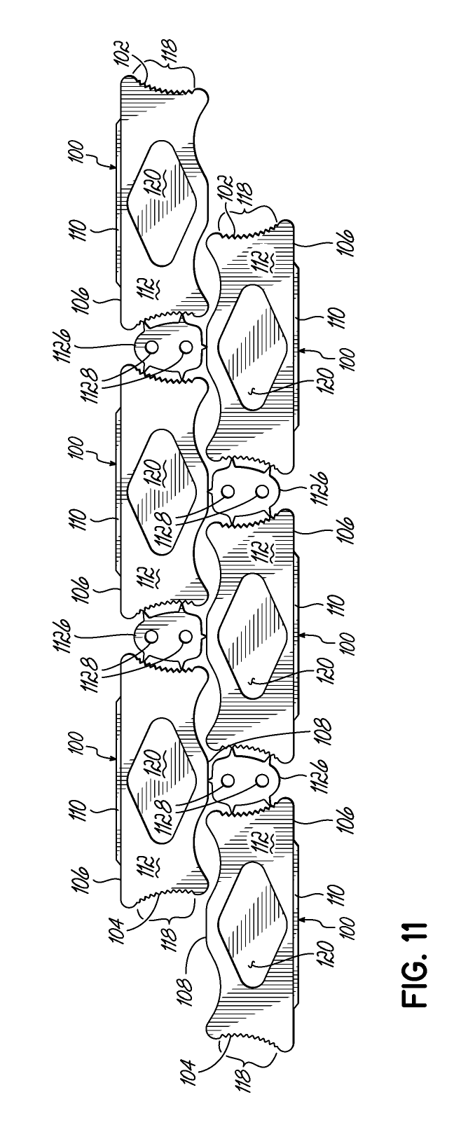

FIG. 11 shows a group of shave biopsy blades 100 at an intermediate step in one possible manufacturing process. The depicted group of shave biopsy blades 100 have been punched, stamped, or otherwise formed from a narrow roll (not shown) of blade thickness 216 sheet metal, and only a portion of the continuous chain of in-progress shave biopsy blades are shown here. As can be seen, the shave biopsy blades 100 are linked together for manufacturing purposes by a plurality of integrally formed holding lugs 1126. The holding lugs 1126 frangibly attach the adjacent first and second side edges 102 and 104 and the back edge 108 of three shave biopsy blades 100. The holding lugs also can include drive holes 1128 (most left unlabeled for clarity) which can be used to feed the strip of attached-together shave biopsy blades 100 through a tractor drive (not shown) to facilitate various manufacturing steps, such as continuous or strip processing.

As an example of a strip processing procedure, the raw blade material may be treated with a photo-resist as is common in printed circuit manufacturing. The strip of blade material is then passed through progressive acid etching baths to chemically remove material. For instance, the bend area feature 120 could be etched into a portion of the thickness of the strip of blade material in one step, with a subsequent step including full removal of the material between adjacent shave biopsy blades 100. Accordingly, a strip of raw blade material can be transformed into a chain of finished blanks which can be further processed in an in-line grinding process that sharpens the blanks by passing them through grinding machines.

As shown in FIG. 11, the bend area features 120 have already been etched into the shave biopsy blades 100, and selected portions of the front edges 106 have been sharpened to create cutting edges 110. Therefore, the depicted shave biopsy blades 100 are nearly complete, with just separation, cleanup, inspection, and packaging tasks remaining. The partial strip of shave biopsy blades 100 shown in FIG. 11 is a non-limiting illustration which depicts one option for manufacturing of the shave biopsy blades, but one of ordinary skill in the art could readily provide other manufacturing procedures or options, depending upon the desired final structure of the shave biopsy blades.

While aspects of the present invention have been particularly shown and described with reference to the preferred embodiment above, it will be understood by those of ordinary skill in the art that various additional embodiments may be contemplated without departing from the spirit and scope of the present invention. For example, the shave biopsy blade 100 could be made of surgical steel such as Hitachi Gin 5 or 440C, any other high tensile strength stainless steel, or any other suitable metallic or nonmetallic material, or combinations thereof, capable of sharpening into a cutting edge 110. The friction-increasing features 118 could be integrally formed with the rest of the shave biopsy blade 100 and/or could include an external friction-increasing agent, such as a rubberized coating. The shave biopsy blade 100 could be provided to the user in at least a slightly pre-bent configuration, rather than having the straight resting configurations of various Figures. The shave biopsy blade 100 could be asymmetrical in those areas currently shown in the Figures as being symmetrical. The shave biopsy blade 100 could form the predetermined bend profile through any suitable combination of plastic and/or elastic bending. A portion of the user's body other than the digits could be used to provide any of the described forces to the shave biopsy blade 100, as could an automatic or manual assistance tool. A device or method incorporating any of these features should be understood to fall under the scope of the present invention as determined based upon the claims below and any equivalents thereof.

While the present invention has been illustrated by a description of various preferred embodiments and while these embodiments have been described in some detail, it is not the intention of the Applicants to restrict or in any way limit the scope of the appended claims to such detail. Additional advantages and modifications will readily appear to those skilled in the art. The various features discussed herein may be used alone or in any combination depending on the needs and preferences of the user. This has been a description of illustrative aspects and embodiments the present invention, along with the preferred methods of practicing the present invention as currently known.

What is claimed is:

1. A unitary shave biopsy blade, comprising:
a first side edge;
a second side edge laterally spaced from the first side edge;
a front edge extending laterally between the first and second side edges;
a back edge extending laterally between the first and second side edges and spaced longitudinally from the front edge, at least one of the front and back edges being at least partially sharpened to form a cutting edge;
a top surface defined laterally between the first and second side edges and longitudinally between the front and back edges; and
a bottom surface defined laterally between the first and second side edges and longitudinally between the front and back edges and being spaced from the top surface by a blade thickness;
wherein at least one of the top and bottom surfaces includes a bend area feature which defines a local reduction in the blade thickness.

2. The unitary shave biopsy blade of claim 1, wherein at least one of the first side edge, the second side edge, and the back edge is ergonomically contoured for contact with a digit of a user.

3. The unitary shave biopsy blade of claim 1, wherein at least one of the first side edge, the second side edge, and the back edge includes a friction-increasing feature for engagement with a digit of a user.

4. The unitary shave biopsy blade of claim 1, wherein the bend area feature is etched into at least one of the top and bottom surfaces.

5. The unitary shave biopsy blade of claim 1, wherein the bend area feature has a footprint shape operative to facilitate at least one desired bending characteristic upon application of compressive force in the lateral direction to the unitary shave biopsy blade.

6. The unitary shave biopsy blade of claim 1, being configured to bend in a lateral direction under compressive force, the location and amount of bending of the blade being responsive to at least one of the amount of compressive force applied; the relative configurations of the first and second side edges, front and back edges, and blade thickness; and a footprint shape of the bend area feature.

7. The unitary shave biopsy blade of claim 1, wherein each cutting edge is laterally separated from the first and second side edges.

8. The unitary shave biopsy blade of claim 1, wherein the bend area feature is spaced apart from the first and second side edges and the front and back edges.

9. A method of excising a target area from a surface of a skin tissue, the method comprising the steps of:
providing a unitary blade including longitudinally oriented and laterally spaced first and second side edges, laterally oriented and longitudinally spaced front and back edges, and oppositely disposed top and bottom surfaces, at least one of the front and back edges being at least partially sharpened to form a cutting edge spaced apart from both the first and second side edges, and at least one of the top and bottom surfaces including a bend area feature which defines a local reduction in a thickness of the unitary blade;
applying a laterally directed compressive force to the unitary blade to bend the unitary blade into a convex relationship with the surface of the skin tissue;
adjusting the compressive force to place the unitary blade into a predetermined bend profile, a shape of the bend profile being at least partially dependent upon a shape of the bend area feature, the bend profile including at least a portion of the cutting edge; and
excising the target area from the skin tissue through substantially longitudinally oriented motion of the unitary blade.

10. The method of claim 9, wherein the step of excising the target area from the skin tissue includes the steps of:
contacting the surface of the skin tissue adjacent the target area with the bend profile;
applying a longitudinally directed motive force to the unitary blade;
moving the unitary blade substantially longitudinally toward the target area;
passing at least a portion of the bend profile through the skin tissue beneath and adjacent the target area to sever the target area with the cutting edge;
moving the unitary blade substantially longitudinally away from the target area; and
removing the unitary blade from the skin tissue.

11. The method of claim 10, wherein the step of applying a longitudinally directed motive force to the unitary blade includes the step of applying a longitudinally directed motive force to at least one of the first and second side edges and the back edge.

12. The method of claim 9, wherein at least one of the first and second side edges of the unitary blade is ergonomically contoured for contact with a digit of a user, and the step of applying a laterally directed compressive force includes the step of grasping each of the first and second side edges with a digit and exerting a laterally directed compressive force thereupon.

13. The method of claim 9, wherein at least one of the first and second side edges includes a friction-increasing feature for engagement with a digit of a user, and the step of moving the unitary blade substantially longitudinally toward the target area includes the step of transferring a longitudinal force from the digit of the user to the unitary blade through the friction-increasing feature.

14. The method of claim 9, wherein the step of moving the unitary blade substantially longitudinally toward the target area includes the step of transferring a longitudinal force from the digit of the user to the unitary blade through the back edge.

15. The method of claim 9, including the steps of:
providing a plurality of unitary blades, with each of the unitary blades having a differently configured bend area feature from the others of the unitary blades; and
selecting a unitary blade from the plurality of unitary blades based at least partially upon the predetermined bend profile of the selected unitary blade provided by the bend area feature thereof.

16. The method of claim 9, including the step of adjusting the predetermined bend profile during excision of the target area by selectively varying the laterally directed compressive force exerted on the unitary blade.

17. A kit comprising a plurality of at least two blades as set forth in claim 1, wherein the respective bend area features are differently configured.

18. The kit of claim 17, wherein the respective bend area features are differently configured in at least one of size, shape or amount of the local reduction in blade thickness.
